(19) United States
(12) Patent Application Publication
(10) Pub. No.: US 2021/0097885 A1
(43) Pub. Date: Apr. 1, 2021

(54) Quantified Movement Feedback System

(71) Applicant: Eric Rosenberg, San Jose, CA (US)

(72) Inventor: Eric Rosenberg, San Jose, CA (US)

(21) Appl. No.: 17/033,731

(22) Filed: Sep. 26, 2020

Related U.S. Application Data
(60) Provisional application No. 62/907,365, filed on Sep. 27, 2019.

(51) Int. Cl. G09B 19/00; A61B 5/11; A61B 5/103; A61B 5/00; G06T 7/20; G09B 5/06

(52) U.S. Cl. CPC G09B 19/003; A61B 5/1128; A61B 5/1038; A61B 5/1124; A61B 5/743; A61B 5/7475; G06T 2207/30196; A61B 5/486; G06T 7/20; G09B 5/065; G09B 19/0015; A61B 2562/0219; G06T 2207/10016; A61B 5/744

(57) ABSTRACT

An analysis system and method for providing movement feedback by sensing and synchronizing different types of information, such as video, inertial and positional information, weight transfer information, audio and music information, etc. The synchronized information is replayed for the user in a manner that enables simultaneous viewing of movement along with calculations and presentation(s) of analysis information related to the movement.

### RELATED APPLICATIONS

This patent application claims priority to provisional patent application 62/907,365, entitled “Method and System for Rule-Based Movement Analysis,” filed on Sep. 27, 2019, and is hereby incorporated herein by reference in its entirety.

### TECHNICAL FIELD

The present invention relates generally to movement analysis systems and more specifically it relates to a quantitative, rule-based, movement analysis system for providing a quick and accurate assessment and analysis on an individual's movement performance and abilities.

### BACKGROUND OF THE INVENTION

Movement analysis systems and devices have been in use for years from video analysis to motion capture to force plates to wearables. However, each type of movement analysis system has its disadvantages.

Video analysis is still subjective, because the analysis is made by the eye of the beholder. Slow motion, “freeze frame,” and other changes to video only help break down the movement; these changes to video only scratch the surface for movement analysis and assessment. They do not provide an extensive analysis of the movement. Video analysis, described above, has been used especially in sports.

There are numerous video analysis systems and devices that have been used. For example, U.S. Pat. No. 5,616,089 to Miller; U.S. Pat. No. 5,363,297 to Larson, et al.; U.S. Pat. No. 9,744,421 to Chan; U.S. Pat. No. 10,380,409 to Thornbrue, et al.; U.S. Pat. No. 8,228,372 to Griffin; U.S. Pat. No. 10,025,986 to Grieb, et al.; U.S. Pat. No. 8,848,058 to Ayer, et al.; all are illustrative of such prior art.

Chan (U.S. Pat. No. 9,744,421) discloses a method, apparatus, system, and computer program for analyzing video images of sports movement. Chan specifically teaches the program to automatically extract segments of the video containing the sports motion. The segment of sports motion is between two of the video image frames showing the key motion positions within the video.

Griffin (U.S. Pat. No. 8,228,372) discloses a digital video editing and playback system and methods of editing and playing back digital videos. Griffin specifically teaches the video processor of the system to receive video segments from multiple sources and to process the video segments. The video processor includes software instruction to evaluate the video segments' synchronization info and form associations with video segments from different sources that correspond to a common event.

In contrast, motion capture and force plates have been beneficial for objective, biomechanics analysis. Since the 90s, motion capture has been used in video games, simulations, choreography, and cinematography. Even, dance movement has thus far been captured and simulated with motion capture systems to create other forms of art and to help aid in dance creation and choreography. Recently, motion capture has been used to study movement and provide insight about movement using biomechanics principles. Motion capture equipment is extremely costly, especially equipment with markerless cameras. For the average individual, motion capture is expensive, robust, lacks mobility, and requires a lengthy period of time for setup and calculation. Motion capture is highly research-driven, not really used as a coaching or feedback tool. However, as sensors have become smaller, cheaper, and lower in power, wearable sensor motion capture systems have grown in popularity. Wearable systems are lower grade of data, but the systems themselves are more affordable and provide for more mobility than most motion capture systems.

There are numerous motion capture systems and devices spanning from wearable sensors to robust systems with markerless camera systems that have been used. For example, U.S. Pat. No. 9,981,193 to Adams, et al.; U.S. Pat. No. 6,685,480 to Nishimoto, et al.; WO2015139145 to Comeau, et al.; U.S. Pat. No. 10,249,213 to Liu, et al.; U.S. Pat. No. 6,437,820 to Josefsson; U.S. Pat. No. 6,415,043 to Josefsson; U.S. Pat. No. 9,885,623 to Drueding, et al.; U.S. Pat. No. 9,679,392 to Richardson; U.S. Pat. No. 9,427,179 to Mestrovic, et al.; U.S. Pat. No. 7,264,554 to Bentley; U.S. Pat. No. 8,165,844 to Luinge, et al.; U.S. Pat. No. 5,344,323 to Burns; U.S. Pat. No. 6,315,571 to Lee; U.S. Pat. No. 9,033,712 to Vasin; U.S. Pat. No. 6,567,536 to McNitt et al. all are illustrative of such prior art.

Bentley (U.S. Pat. No. 7,264,554) discloses a system and method for analyzing and improving the performance of an athletic motion such as a golf swing. Bentley specifically teaches the system to provide a real-time, information rich, graphic display of the results in multiple, synchronized formats including video, color-coded, and stepped frame animations from motion data, and data/time graphs. Based on the results, a user-specific training regime with exercises are selected. To produce such results, a user's movements is monitored with instrumented inertial sensors and video cameras.

Luinge, et al. (U.S. Pat. No. 8,165,844) discloses a system of motion sensor modules placed on various body segments to capture the movement of an object. The sensor modules capture three-dimensional inertial data relating to their respective body segments. Luinge, et al. specifically teaches the sensor modules to process the sensor data through digital signal processing filters and biomechanics constraints to estimate orientation and position of the corresponding body segments.

Vasin (U.S. Pat. No. 9,033,712) discloses an invention and training method for comparing digitized movement to a reference movement. Vasin specifically teaches the computer to compare the digitized movement with the reference movement of an expert or computer simulation and to control tactile feedback elements to perform the correction action. If the trainee deviates from the reference movement, then the tactile action is received. The device includes sensors for on-line movement digitizing.

McNitt, et al. (U.S. Pat. No. 6,567,536) discloses an analysis system and method for providing athletic training and instruction. McNitt, et al. specifically teaches the system to sense and to replay synchronized information, such as video, positional information, and weight transfer information, for the user and to allow for simultaneous viewing along with calculations and analysis related to the athletic motion.

While these inventions may be suitable for the particular purpose to which they address, they are not suitable for providing an accurate assessment and analysis on an individual's movements based on correct models of movement in order to improve an individual's craft. The current movement feedback systems only analyze certain, specific movement(s), and do not provide movement feedback associated with synchronized, rhythm and timing analysis.

In this respect, the proposed movement analysis system departs substantially from the conventional methods of use and compositions of the prior art. In doing so, the present invention provides a composition and a method of using the composition primarily developed for the purpose of providing a quick and accurate assessment and analysis of an individual's movements.

### SUMMARY OF THE INVENTION

The invention is inspired from the field of dancing, specifically ballroom dancing, but it is to be understood that the proposed invention is not limited to one field, industry, or application. The invention is not limited by the individual's expertise in their chosen field. Any person with any level of expertise can benefit from the proposed invention. The invention is capable of other embodiments and of being practiced and carried out in various ways. Also, it is to be understood that the phraseology and terminology set forth are not to be regarded as limiting.

In light of dancing, as the inspiration of the proposed invention, whatever the reason or goal might be, athletes, including ballroom dancers, work on their craft to achieve perfection. It is no understatement when a coach tells their athletes, “Practice makes perfect.” To even just taste what perfection is, an individual needs to overcome themselves to endure hard efforts of repetition of the same movements over and over again. Particularly, dancers become the best by repeating the same, fundamental figures of movement to music over and over again.

Movement is copied and has been copied from our ancestors and will be passed down to future generations. In the scope of dancing, dance instructors teach and guide their students, and the students then copy. Humans are the best imitators, but copying movement is very difficult. Rhythmic movement or dance movement is particularly even more challenging to copy because there are both technical and artistic requirements to it.

Articulating movement is even more difficult. The information described from movement experts and professionals may be the same. Yet, the difference lies in how the information is expressed. Movement is visual. Passing along and perceiving the information about movement is a combination of visual and verbal learning, and within dancing, there is a third learning style, aural learning. The individual receiving the information needs to rely on their own imagination to then articulate and understand the same message that was articulated by the movement expert who was once in the shoes of the individual receiving the information. Movement training is very dimensional and requires a great skill of communication on behalf of both parties: the expert and the receiver.

There exists a perpetual cycle of misinterpretation between the expert and receiver. As a result, feedback individuals receive regarding their movement can be vague, contradictory, interpreted incorrectly, and/or not sufficient enough for individuals to learn and improve their movement potential and performance.

Also, the receiver cannot see what the expert sees, and even the experts cannot analyze all of the minute movements and transitions of the receiver from all directions in real time. That leaves room for human error. Henceforth, dancing or any movement-based activity has become more and more difficult to assess and judge.

There are also challenges associated with judging that should be addressed. In ballroom dancing, there are two international governing bodies: World Dance Council (WDC) and World Dancesport Federation (WDSF). Almost ten years ago, WDSF created a new judging system called the Judging System. This new judging system is based on a 1-10 scale. 1 on the scale is very poor, and 10 on the scale is outstanding. The Judging System is only practiced in World Championship events. For local, regional, and national competitions, the judging system is not practiced. The new Judging System for the WDSF organization provides zero feedback because the scale is subjective. No one in the ballroom dancing industry has bothered to assess the problem of insufficient and subjective assessment of dance movement, except Bologna State University has done research in coordination with a dance studio and dance team in Italy, Team Diablo, to assess movement with motion capture. However, there is still no movement analysis system for ballroom dancers to increase their performance. Judging is just as subjective as coaching.

During practice, dancers use mirrors or video recordings to assess their own performance. The assessment is very subjective. When coaches instruct their dance students, students focus on a specific dimension of their dancing, and the eye can only see so much. When a dancer stands in front of a mirror, the dancer only sees the body parts that are being reflected back from the mirror to the dancer's eyes. Dancers are limited in ways to assess their own dancing, just like their coach(es). The typical coach in the dance industry is not so different from the student; they are still a student, just with more experience.

Any individual who practices movement-based activities have a common goal: to improve and perfect their performance over time. Clearly, there is a need for a rule-based, quantitative movement analysis system to accurately assess and analyze movements based on correct models of movement in order to improve an individual's craft.

In view of the foregoing disadvantages inherent in the known types of movement analysis devices and systems present in the prior art, the present invention provides a new quantitative and rule-based movement analysis system wherein the same can be utilized to provide a quick and accurate assessment and analysis of an individual's movements.

The general purpose of the present invention, described subsequently in greater detail, is to provide a new movement analysis and feedback system that has many of the advantages of the movement analysis systems mentioned heretofore and many novel features and functions that result in a new movement analysis and feedback system which is not anticipated, rendered obvious, suggested, or even implied by any of the prior art movement analysis systems, either alone or in any combination thereof.

To attain this, the present invention generally comprises the process of recording and capturing the video and data synchronously during an individual's movement training, transferring the collected data in real time or after data collection to a central computing entity through a communication system, entering the data into the central computing entity database, conducting a movement analysis of the collected data in real time or after data collection at the central computing entity, determining the results of the movement analysis in real time or after data collection, transferring the results to the smart device in real time or after data collection through a communication system, and displaying the results upon a display monitor of the smart device in real time or after movement recording and data collection. Based upon the displayed results, any individual can then make any improvement(s) necessary to better their movement. The results of the movement analysis may include synchronized, detailed, and color-coded annotations, animations, and comments of movement and audio feedback layered over the video. The results of the movement analysis are calculated utilizing biomechanics principles and novel movement interpretation algorithms. It can be appreciated that the present invention may be utilized to analyze any movement. The present invention allows for the individual to give access to the results from the movement analysis to any individual around the world for additional consultation(s). The total number of smart devices capable of communicating with the central computing entity is virtually unlimited thereby allowing unlimited access of movement feedback information upon user access.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a movement analysis and feedback system that will overcome the shortcomings of the prior art.

Another object is to provide a movement analysis and feedback system that provides an accurate assessment and analysis of an individual's movement.

An additional object is to provide a movement analysis and feedback system that allows any individual to give access to the results of their movement analysis to any individual around the world for additional consultation(s).

A further object is to provide a movement analysis and feedback system that increases the accuracy of the assessment of the individual's movement.

An additional object is to provide a movement analysis and feedback system that synchronizes the data beginning to end of the data collection.

A further object is to provide a movement analysis and feedback system that provides an assessment based upon data collected in real time or immediately after finishing data collection.

A further object is to provide a movement analysis and feedback system that instantly displays the movement feedback and assessment in an easy to understand format for an individual to improve their movement.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific use illustrated and described within the scope of the appended claims.

### BRIEF DESCRIPTION OF THE DRAWINGS

Various other objects, features and attendant advantages of the present inventions will become fully appreciated as the same becomes better understood when considered in conjunction with the accompanying drawings, in which like reference characters designate the same or similar parts throughout the several views, and wherein:

FIG. 1 is a diagram of a movement analysis system.

FIG. 2 is a criteria sample for movement analysis, specifically dynamic foot pressure analysis.

FIG. 3 is the criteria for defining the criteria in FIG. 2.

FIG. 4 is a mobile embodiment of an analysis system, employing wireless body sensor modules in accordance with embodiment of invention.

FIG. 5 is a sample overview of dynamic foot pressure analysis results with timing analysis results for a ballroom dancer's rumba walks.

FIG. 6 is a sample scoring of dynamic foot pressure analysis results for a ballroom dancer's rumba walks.

FIG. 7 is a sample display of the movement feedback results.

FIG. 8 is a flowchart that illustrates the operational characteristics related to control of the data collection of the present invention.

FIG. 9 is a simplified flowchart that illustrates the functional components of the processing and analysis stages of the present invention.

FIG. 10 is a flowchart for any analysis category of the present invention utilizing pressure data.

FIG. 11 is a flowchart for any analysis category of the present invention utilizing motion capture data and video data.

FIG. 12 is a flowchart for the timing analysis of the present invention.

FIG. 13 is a flowchart for displaying results of the present invention.

### DETAILED DESCRIPTION OF EMBODIMENTS

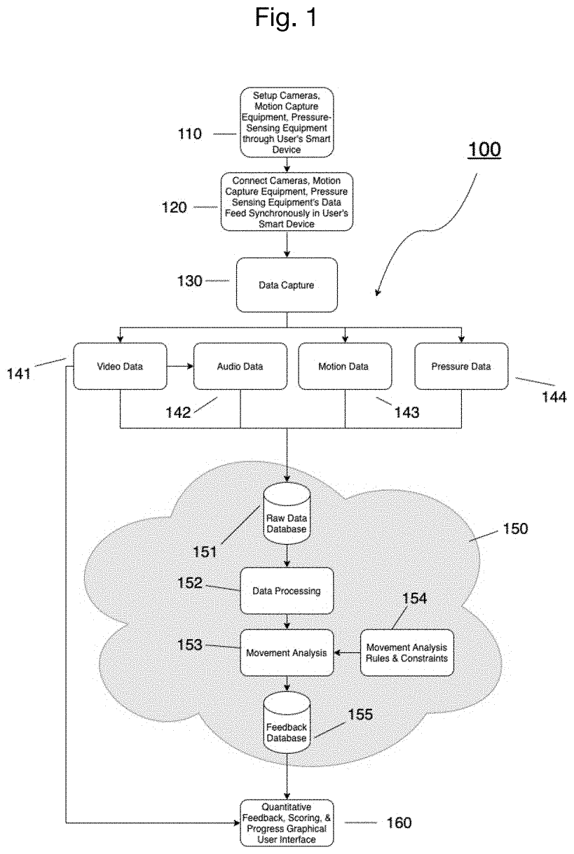

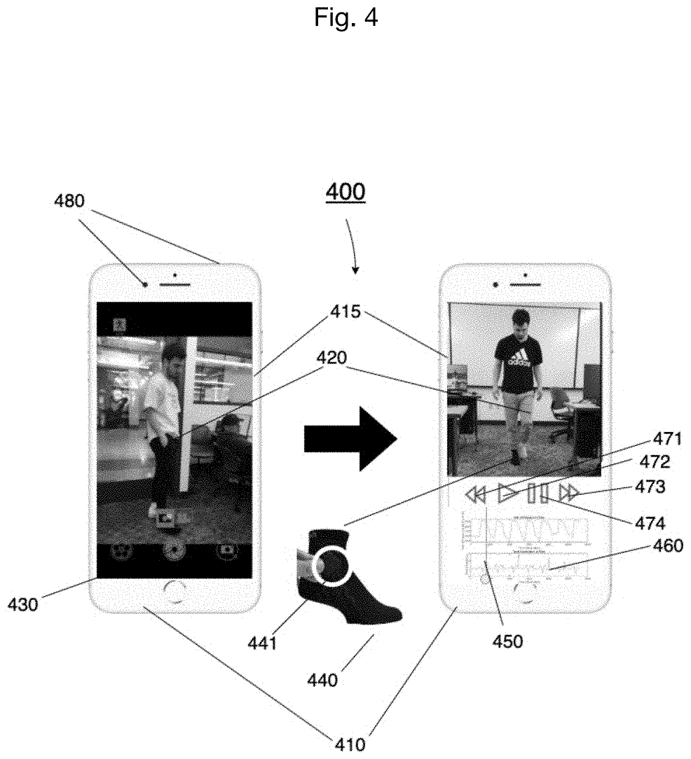

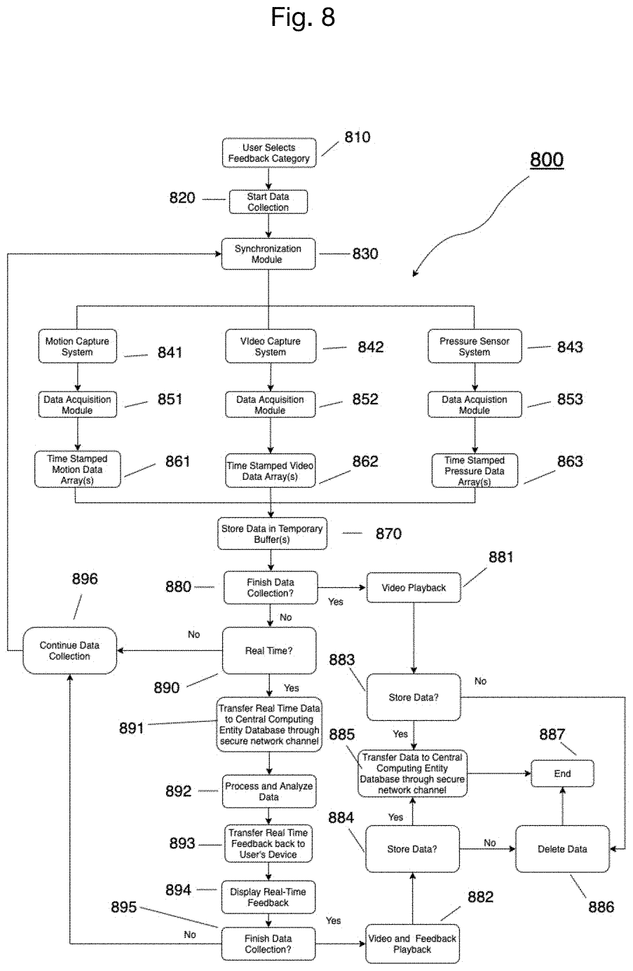

Turning now to a detailed description of the drawings and embodiments, it is noted that similar reference characters denote similar elements throughout the several views, where FIGS. 1 through 13 illustrate a movement analysis system and method according to a preferred embodiment of the present invention. FIG. 1 shows an overview of the movement analysis system (100) of the present invention. Description of the exemplary movement analysis system (100) of FIG. 1 will include reference to elements shown elsewhere in FIGS. 4, 7, 8, and 13, for example, where those elements and figures will provide further detail. The movement analysis system (100) comprises the processes of recording and capturing movement data including (130) video (141) and audio (142) with inertial measurement data (143), pressure data (144), and reflective marker data (143) synchronously (using a synchronization module (830) as shown in FIG. 8) during an individual's movement training. This is followed by transferring the collected data (141,142,143,144) in real time (891) or after data collection (885) to a central computing entity (150) through a communication system or network such as the Internet, entering the collected data (141,142,143,144) into a central computing entity database (151) for the user's raw collected data (141,142,143,144), processing (152) the raw collected data (141,142,143,144) in the central computing entity (150), conducting a movement analysis (153) of the collected data (141,142,143,144) in real time (890) or after data collection (820) in the central computing entity (150), storing the results (1320) of the movement analysis in real time (890) or after data collection (820) into the central computing entity database (155) for the user's movement feedback, transferring (893,1310) the results (1320) to a smart device (410) in real time (890) or after data collection (820), and displaying (1330,894) the results (1320) upon a display monitor (160) of the smart device (410) in real time (890) or after movement recording and data collection (820).

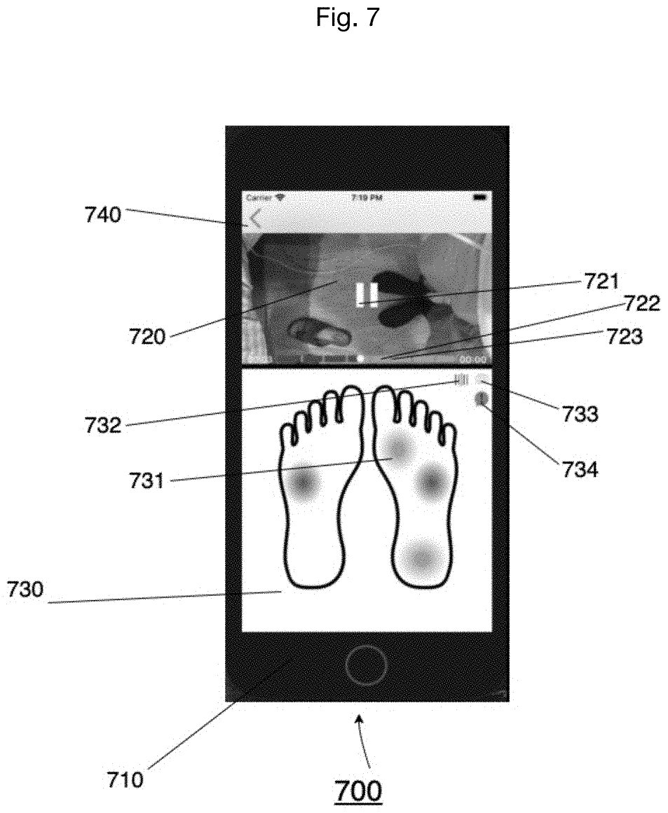

Based upon the displayed results (1320), any individual can then make any improvement(s) necessary to better their movement. With reference to a sample display of the movement feedback results shown in FIG. 7, the results (1320) of the movement analysis (153) may include synchronized, detailed, and color-coded annotations (723,731), animations (720,730), and comments (734) of movement and audio feedback (723) on a scrubber (722) layered over video playback (720). The results (1320) of the movement analysis are calculated utilizing biomechanics principles and novel movement interpretation algorithms (154).

It can be appreciated that the present invention may be utilized to analyze any movement. The present invention allows for the individual to give access to the results from the movement analysis to any individual around the world for additional consultation(s). The total number of smart devices capable of communicating with the central computing entity (150) is virtually unlimited thereby allowing unlimited access of movement feedback information upon user access. The above process will now be described in greater detail.

Exemplary Movement Data Capture and Analysis System

FIG. 4 is a block diagram of an exemplary mobile movement data capture and analysis system (400) for practicing the various aspects of the present invention. Preferably garment(s)(440) and/or a body suit are intended to be worn for collecting motion (143) and pressure data (144) from a user (420), accompanied with video data (141) and audio data (142) from camera footage (480). However, in other embodiments, sensor modules (441) do not have to be associated with garment(s) (440) or a body suit.

The sensor modules (441) may also include pressure sensors in the soles of shoes or on the bottom of socks (440), thereby collecting pressure data (144). The sensor modules (441) may also capture three-dimensional (3D) position, orientation, and inertial data from three-dimensional inertial measurement sensors in respect to the body segments, thereby gathering motion data (143) having six degrees of freedom with respect to a coordination system not fixed to the body. Additional embodiments may include using a plurality of cameras (480) to aid in obtaining 3D motion data (143), video data (141), audio data (142), and for visualizing the movement analysis results (1320) over video (141).

The exemplary synchronization module (830) synchronizes physical motion (143), pressure (144), video (141), and audio (142) data received from the sensor module (441) and cameras (480) and communicates through a network interface the resulting synchronized information (861,862,863) to a processing module (152) in the central computing entity (150). The data processing module (153) of the central computing entity (150) includes a processor for executing stored instructions and a memory for storing instructions to be executed by the processor.

In some embodiments, all collected data samples, whether video (141), audio (142), motion (143), pressure (144) or any other sample associated with the movement to be analyzed, are timestamped using the same timebase. In this embodiment, timestamps are administered on each information signal and on preset intervals such that the corresponding samples of the signals are identified by the same timestamp. In another embodiment, the information signals might be time-stamped using an internal clock mechanism. Accordingly, each sample from the first information signal corresponds to a sample from the second and all other information signals. Time stamping the information signals creates synchronized information that is transmitted to the processing module (152) to provide synchronized movement analysis (153) associated with the information acquired by the sensors (441), markers, and cameras (480).

The exemplary processing module (152) receives synchronized information (861,862,863) from the synchronization module (830) and, in turn, processes the synchronized information (861,862,863) in order to provide the movement feedback results (1320) from the movement analysis (153) to an end user (420). In accordance with this embodiment, the central computing entity's databases (151,155) store the value of each timestamp with each data sample (141,142,143,144).

The exemplary movement analysis (153) is then used to provide the movement feedback results (1320). The movement feedback results (1320) are in a form suitable for review by the user (420). In accordance with the preferred embodiment, such movement feedback results (1320) are transferred back to the user (420) through the network channel to a graphical user interface (160) operating on a display (415) of the user's smart device (410).

A network interface circuit (not shown) may be utilized to send and receive data over a network connected to other smart devices (410). An interface card or similar device (not shown) and appropriate software implemented by a microprocessor in the user's smart device (410) can be utilized to connect the smart device (410) to an existing network and transfer data according to standard protocols, such as WiFi.

The present invention is preferably operated upon a global computer network such as the Internet. The Internet is understood to comprise a global computer network providing a variety of information and communication facilities, consisting of interconnected networks using standardized communication protocols. As such, a plurality of computer systems around the world are in communication with one another via this global computer network. The present invention preferably utilizes the Internet and related communications protocols, as well as Bluetooth, a standard for the short-range wireless interconnection of mobile phones, computers, and other electronic devices; however, it can be appreciated that as future technologies are created that various aspects of the invention may be practiced with these improved technologies. More particularly, wireless technologies provide a suitable medium for operating the present invention.

The display screen (415) is preferably an input/output device that displays images, comments, videos, annotations, animations of data provided by the microprocessor via the peripheral bus or provided by other components in the smart device (410). Other types of user input devices can also be used in conjunction with the present invention. However, it can be appreciated that as future technologies are created that various other input devices, like augmented or virtual reality devices, may be used in the present invention.

Step 1a. Data Acquisition

When any individual wants to acquire feedback of their own or another individual's movement, the individual moving may put on motion capture equipment and pressure sensing equipment accompanied by cameras (480). The exemplary mobile movement data capture and analysis system (400), with reference also to the overall system of FIG. 1 and the processes of FIG. 8, provides a user (420) means to capture (130) data on movement and to process (152) and analyze (153) that data. The individual user (420) will check to make sure all the sensors (441) are working properly and all connections (120) and communications are intact and provided for in an initial setup (110) on the smart device (410) for data capture (130). Also, if the user (420) has multiple cameras (480) set up in their environment, the cameras (480) will also need to be checked to make sure all cameras (480) are working properly and all connections (120) and communications are intact. If the user (420) chooses to use a smart device (410) like a smartphone or tablet for mobility purposes rather than a desktop or laptop computer, another user will be required to control the smart device (410). After the setup (110) is complete, the user (420) will have an option to select a custom countdown or choose to manually start or stop the data collection (820), and also select which category or categories (810) of movement feedback the user wants analyzed.

On the display (415) of the smart device (410), the user (420) or another individual will click or touch a record button (430) to begin data collection (820), where the data collection synchronizes, via the synchronization module (830), data from a video capture system (842), with data from a motion capture system (841), and a pressure sensor system (843), as shown in FIG. 8, by means of a timestamp. During data capture (130), synchronized and timestamped video data (862), motion data (861), and pressure data (863) is processed through a data acquisition module (852,853,851) and then stored in temporary data arrays or buffers (870) until the data capture (130) has finished. If a custom countdown is set, the recording will stop when the countdown is finished. If a custom countdown is not selected, the user (420) or another individual will need to manually click or touch a record/stop button (430) to stop recording (880) and finish data collection. When the recording is finished, the user (420) will have an opportunity to playback video (881) of the movement and decide whether the user wants to store (883) and transfer (885) the collected and synchronized data (141, 142, 143, 144) to the central computing entity (150) through a secure network channel or delete (886) the data to complete (887) the process and return to collecting new data (820).

On the display (415) of the smart device (410), after data collection, there will be displayed play (472), rewind (471), forward (473), and pause (474) buttons for video playback (881), as well as a save (883) and trash (886) icon/button. If the trash (886) icon is selected, the display (415) will return to the user's dashboard where the user (420) can go back and select the data capture (130) mode to repeat the process of data collection (800). If the save (883) icon is selected, the user (420) will have the option to title their log, or the system (100, 400) will automatically provide a timestamp of the logged data as the log title. After titling the logged data (141,142,143,144), the logged data (141,142,143,144) will be uploaded and sorted directly to a user's folder in the central computing entity database (151) through a communication channel. Shortly after, the display (415) will return to the user's dashboard where the user can go back and select the data capture (130) mode to repeat the process of data collection (800) or review (1300) previous results or awaiting results.

If the movement analyzed is accompanied with music, audio (142) will be extracted (1200) for analysis from the recorded video.

Step 1b. Data Acquisition and Displaying Results in Real Time

When any individual wants to acquire feedback in real time (890) of their own or another individual's movement, the individual moving may put on motion capture equipment and pressure sensing equipment accompanied by cameras (480). The individual will check to make sure all the sensors (441) are working properly and all connections (120) and communications are intact and provided for in the initial setup (110) on the smart device (410) for data capture (130). Also, if the user (420) has multiple cameras (480) set up in their environment, the cameras (480) will also need to be checked to make sure all cameras (480) are working properly and all connections (120) and communications are intact. If the user (420) chooses to use a smart device (410) like a smartphone or tablet for mobility purposes rather than a desktop or laptop computer, another user will be required to control the smart device (410). After the setup (110) is complete, the user (420) will have an option to select a custom countdown or choose to manually stop the data collection (820), and also select which category or categories (810) of movement feedback the user (420) wants analyzed.

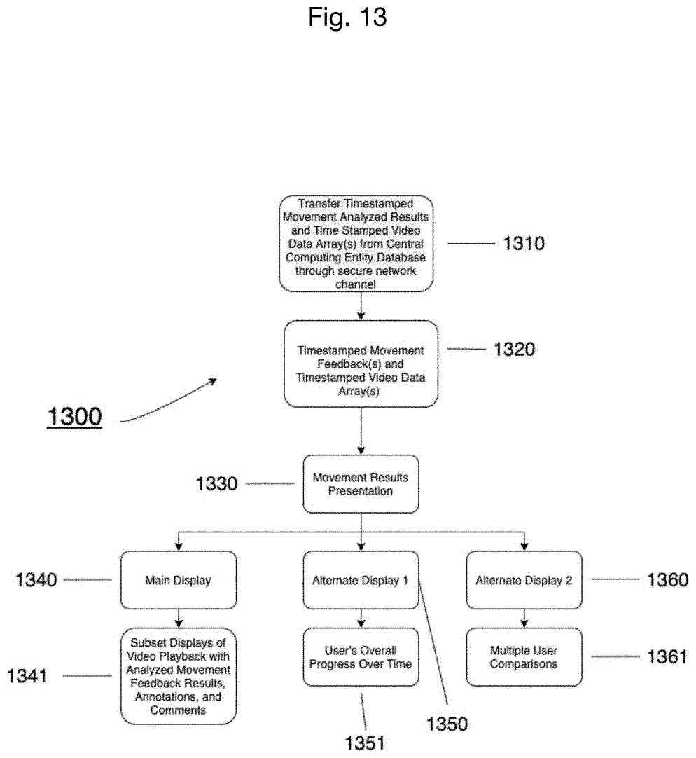

On the display (415) of the smart device (410), the user (420) or another individual will click or touch a real time record button (430) to begin the data collection program (820), which synchronizes via the synchronization module (830) the video capture system (842) with the motion capture system (841) and pressure sensor system (843), as shown in FIG. 8, by timestamp. During data capture (130), the synchronized and timestamped video data (861), motion data (862), and pressure data (863) is processed through the data acquisition module (852) and then stored in temporary data arrays or buffers (870) which are then immediately transferred to the central computing entity (891), where the feedback analyses program(s) immediately process and analyze the data (892) and immediately transfers (893) and displays the dynamic movement feedback results (894) in real time with the current video recording on the display (415) of the user's smart device (410) until the data recording has finished (895), as shown in FIG. 13. If the custom countdown is set, the recording will stop when the countdown is finished. If the custom countdown is not selected, the user (420) or another individual will need to manually click or touch the start/stop button (430) to stop recording. When the recording is finished (895), the user (420) will have an opportunity to playback the video with the feedback of the movement (882) and decide whether the user (420) wants to store (884) and transfer (885) the collected data (141,142,143,144) to the central computing entity (150) through a secure network channel or delete (886) the data to complete the process (887) and return to collecting new data (820).

On the display (415) of the smart device (410), after data collection (820), there will be a play (472), rewind (471), forward (473), pause button (474), and playback time scroller (450) for the video playback and playback of the synchronized data (460), as well as a save (884) and trash (886) icon/button. If the trash (886) icon is selected, the display (415) will return to the user's dashboard where the user (420) can go back and select the data capture mode (130) to repeat the process of data collection (800). If the save icon (884) is selected, the user (420) will have the option to title their log or the system (100, 400) will automatically provide a timestamp of the logged data (141,142,143,144) as the log title. After titling the logged collected data (141,142,143,144), the logged data (141,142,143,144) will be uploaded (885) and sorted directly to the user's folder in the central computing entity's database (151) for raw data (141,142,143,144) through a communication channel. Shortly after, the display (415) will return to the user's dashboard where the user (420) can go back and select the data capture mode (130) to repeat the process of data collection (800) or review (1300) previous results or awaiting results.

If the movement analyzed is accompanied with music, audio (142) will be extracted (1200) for analysis from the recorded video (141).

Step 2. Data Entry

After the collected data (141,142,143,144) is acquired, the user (420) will have the option to title their log or the system (100, 400) will automatically provide a timestamp of the logged collected data (141,142,143,144) as the log title. After titling the logged collected data (141,142,143,144), the logged data (141,142,143,144) will be uploaded (885) and sorted by timestamp directly to the user's private folder in the central computing entity's (150) database (151) for raw data (141,142,143,144) through a communication channel.

In many embodiments, no collected data (141,142,143,144) will be stored on the user's smart device (410). All collected data (141,142,143,144) will be stored in the central computing entity (150). The collected data (141,142,143,144) of a user (420) will be stored and sorted in the central computing entity's databases (151,155).

Step 3. Collected Data Transferred to Central Computing Entity

After the user (420) has chosen to save (883,884) the collected data (141,142,143,144), the collected data (141,142,143,144) is then transferred (885) to the central computing entity (150) (cloud storage) through a communications channel. The central computing entity (150) is comprised of a database (151) for storing the raw collected data (141,142,143,144), a data processing module (152), movement analyses programs (153), movement analysis rules and constraints (154), and a database (155) for storing the movement feedback results (951,952,953).

A suitable communications system for the collected data (141,142,143,144) to be transferred upon is the Internet. It can be appreciated that various other well-known communication systems may be utilized for transferring the collected data (141,142,143,144) to the central computing entity (150).

Step 4. Analysis of Collected Data

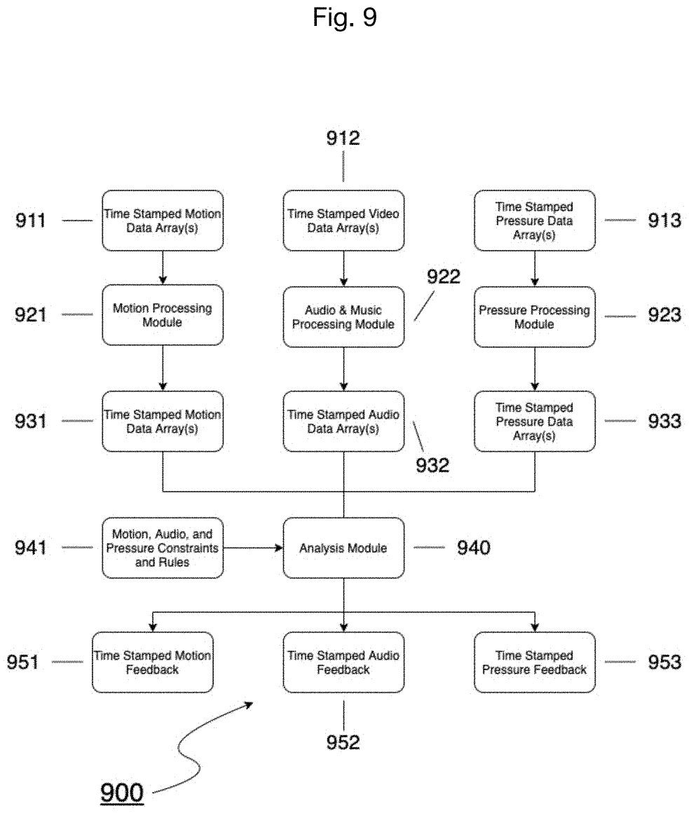

FIG. 9 shows the functional components of the processing and movement analysis stages (900) of the present invention. After the collected data (141,142,143,144) is transferred (885,891) to the central computing entity (150), the central computing entity (150) takes the timestamped motion data array(s) (911), timestamped video data array(s) (912), and timestamped pressure data array(s)(913) for processing (921,922,923). Each data type (motion, pressure, and video) have their own unique processing (931,932,933) in order for the analysis to take place. Once the processing is complete, the specific feedback analyses (940) chosen by the user (811) at the beginning of the data collection process are computed utilizing novel algorithms, established biomechanics formulas, and motion, audio, and pressure constraints and rules (941) to provide the movement analysis results (951,952,953), as shown in FIG. 9.

It is not to be assumed that the processed data (931,932,933) from the pressure sensing equipment, inertial measurement unit sensing equipment, or reflective markers can provide feedback or insight of internal bodily activity. However, the processed data (931,932,933) from the pressure sensing equipment, inertial measurement unit sensing equipment, or reflective markers can also provide for models and simulations of the individual's internal musculoskeletal activities.

The central computing entity (150) includes at least one memory for storing instructions and one processor for executing instructions that is configured to analyze (153) the collected data (141,142,143,144) using one or more movement feedback programs (1000, 1100, 1200). The movement feedback programs can be separated into eight different movement categories: (A) dynamic foot pressure analysis, (B) rotational movement analysis, (C) flexion/extension movement analysis, (D) abduction/adduction/circumduction movement analysis, (E) dorsiflexion/plantar flexion movement analysis, (F) supination/pronation movement analysis, (G) protraction/retraction/depression/elevation/superior/inferior movement analysis, and (H) inversion/eversion movement analysis. Utilizing these individual categories of analysis and combinations of categories of analysis, an accurate assessment can be made of the individual's movement for improvements. Depending on the movement and goal(s) for acquiring movement feedback and analysis, not all analysis categories will be required or combination(s) of analysis categories will be required to entirely assess the movement, because certain movements require use of multiple areas of the body, like in dancing.

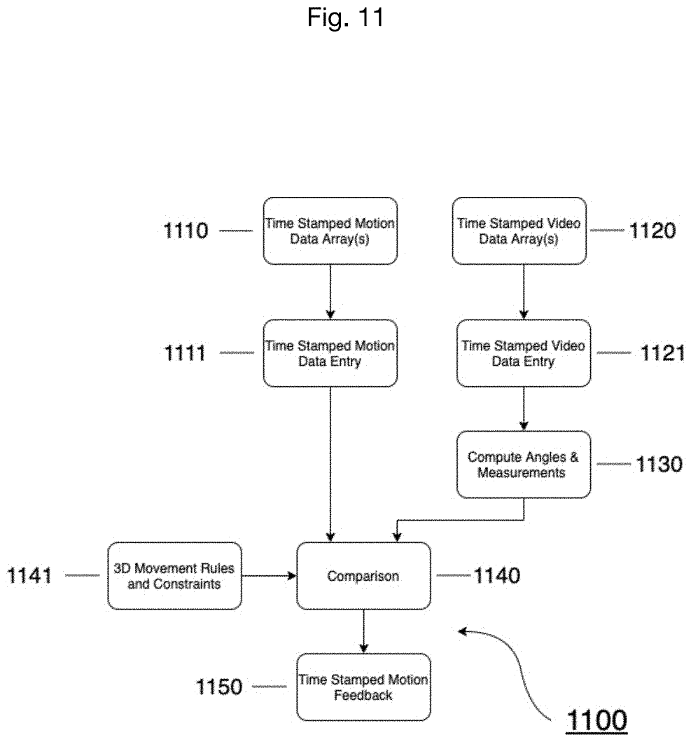

FIG. 11 shows a flowchart for analysis categories (B-H) utilizing video (141) and motion capture data (143). For overview of these analysis categories (B-H), the timestamped motion feedback results (1150) are produced by comparing (1140) each individual motion datapoint (1111) of the timestamped motion data array(s) (1110) with computed angles and measurements (1130) from each individual video frame (1121) of the timestamped video data array(s) (1120) to a model with three-dimensional movement rules and constraints (1141), which represents the ideal physical, three-dimensional movement for the specific movement category or categories for a given activity.

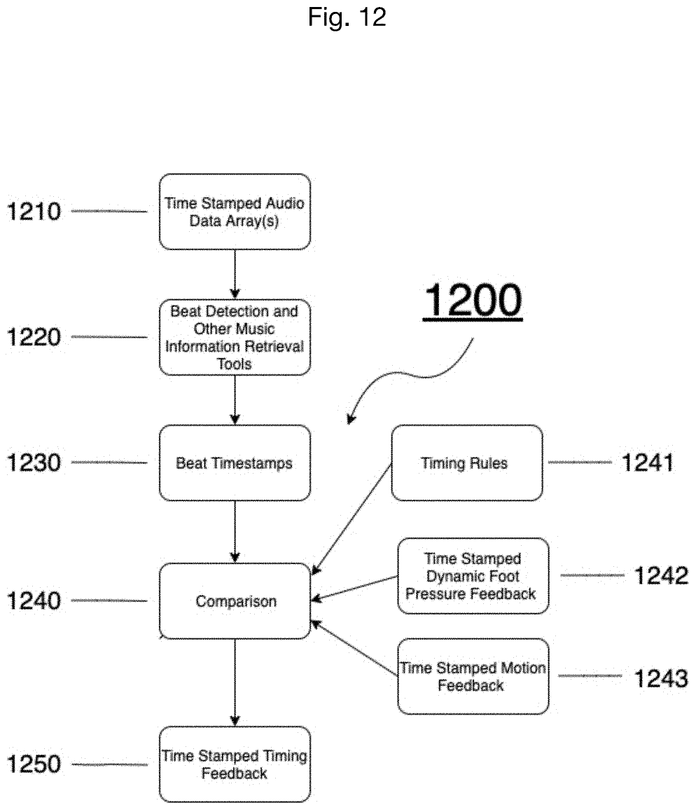

Also, if the movement is accompanied with music, an additional category: (I) timing (1200) is analyzed and compared (1240) with the movement analysis (1242,1243) to provide for an accurate assessment (1250) of the individual's movement to music. FIG. 12 shows a flowchart overview of the timing analysis process.

A. Dynamic Foot Pressure Analysis

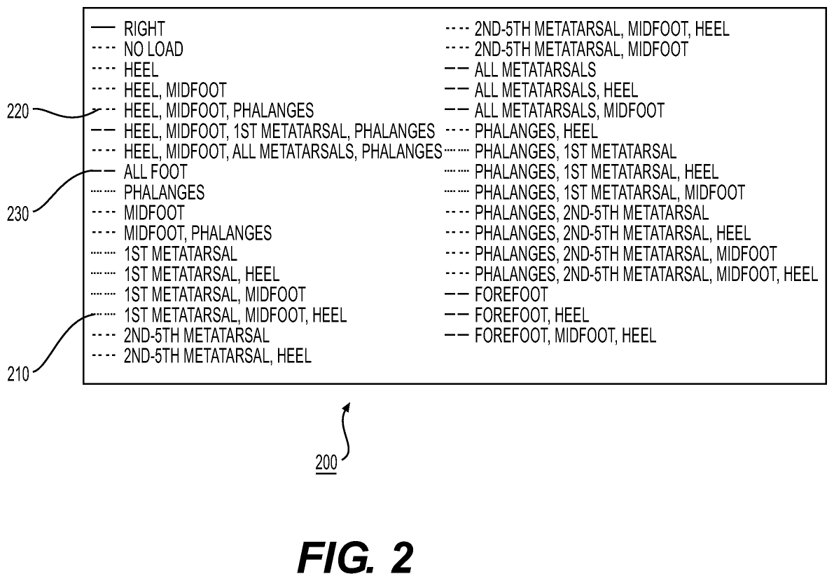

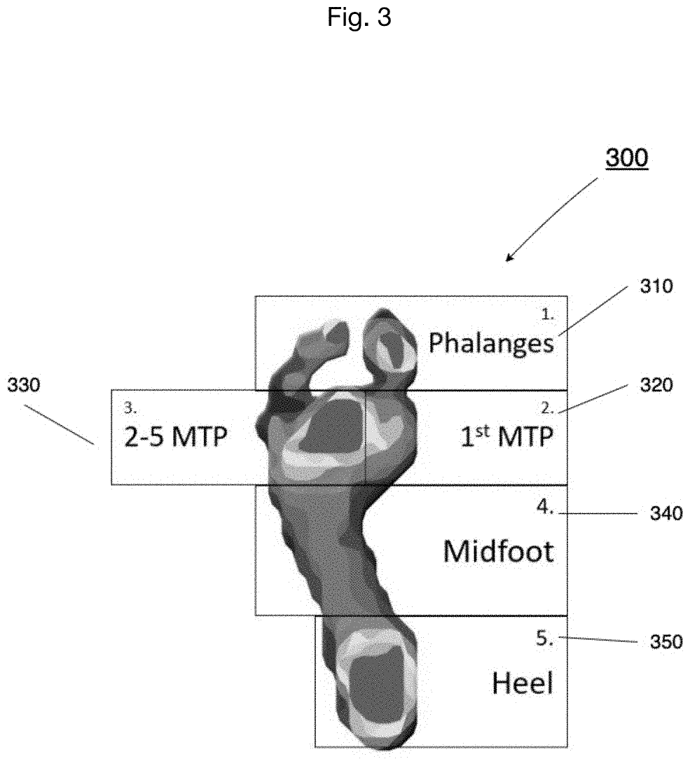

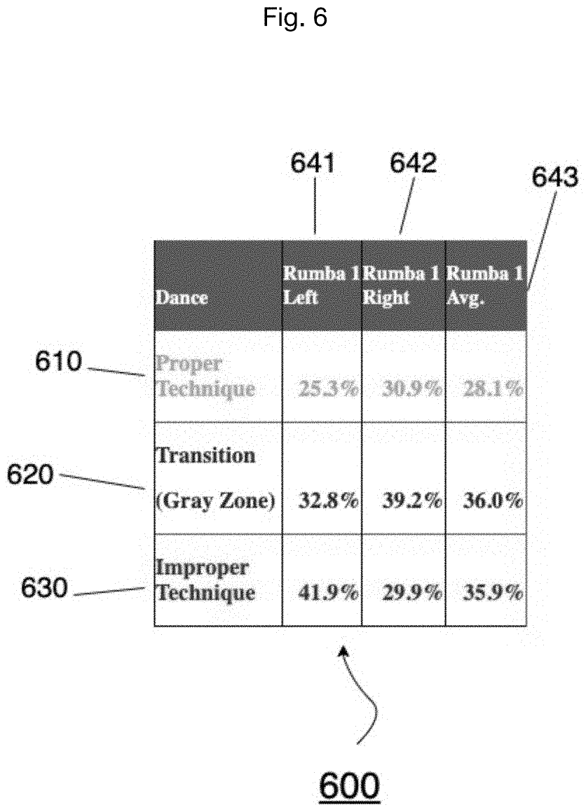

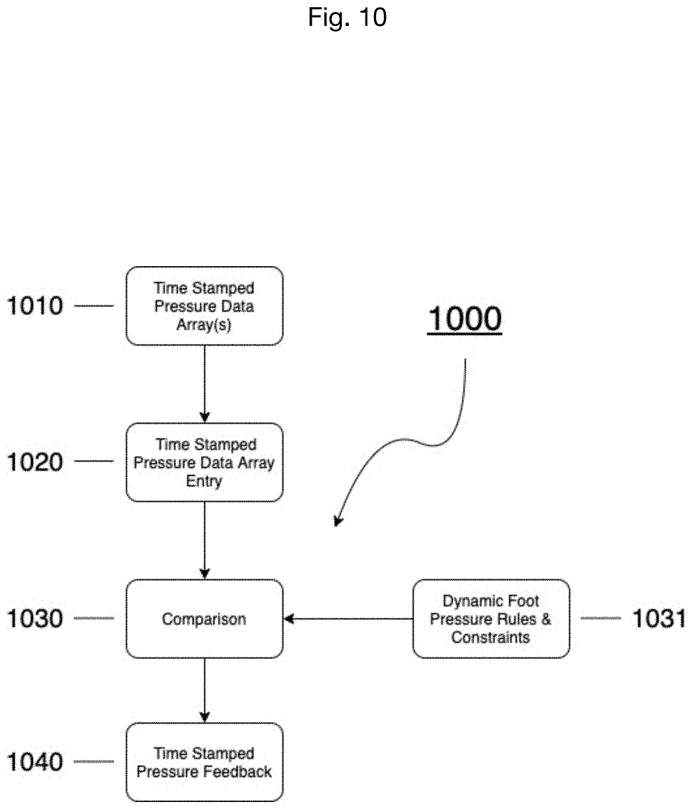

FIG. 10 shows the process of providing movement analysis utilizing pressure data (144). Further reference to FIG. 6, a sample scoring of dynamic foot pressure analysis results (600), is also made in this analysis, as well as to FIGS. 2 and 3, detailing a criteria sample for movement analysis, specifically dynamic foot pressure analysis. The Dynamic Foot Pressure Analysis program (1000) is designed to evaluate (i) the locations (310,320,330,340,350) of pressure of the foot sole during movement at each given timestamp (1020) from the data collection (1010), and further (ii) depending on the movement, the correct (610) and incorrect (630) footwork and transitions (620) in footwork defined by the locations (310,320,330,340,350) of the pressure of the foot sole, utilizing the processed pressure data (1010) of pressure sensing equipment (sensors 441) worn by the individual acquiring movement feedback analysis.

For example, if the movement is dancing, specifically Latin Dancing, the dynamic foot pressure rules and constraints (1031) specify that the pressure on the bottom of the foot during movement should always be on the inside edge of the foot and the ball and toes of the foot never leave the ground. FIG. 2 shows all 32 possible combinations (200) for proper (610) dynamic foot technique in green (210), improper foot (630) technique in red (220), and weight transfers/ambiguous (620) dynamic foot movement in grey (230), based on the five locations (310,320,330,340,350) of the foot (300) shown in FIG. 3. Fewer or more combinations can be addressed in the Dynamic Foot Pressure Analysis program as necessary. The determining factor is the number of pressure sensing elements. Five pressure sensing elements will produce 32 combinations. The number of total possible combinations of dynamic foot pressure movement can be calculated by taking the number of pressure sensing elements (n) as a power of 2 (2n). FIG. 10 shows a flowchart for analysis utilizing pressure data (144). The dynamic foot pressure feedback results (1040) are produced by taking each individual datapoint (1020) of the processed, timestamped pressure data array(s) (1010) and comparing (1030) each timestamped datapoint (1020) to datapoints associated with a foot pressure models stored in the central computing entity (150) that includes predetermined dynamic foot pressure rules and constraints (1031) that include or represent proper (610) dynamic foot technique, improper foot (630) technique, and weight transfers/ambiguous (620) dynamic foot movement for a given activity at a given moment in time during the movement.

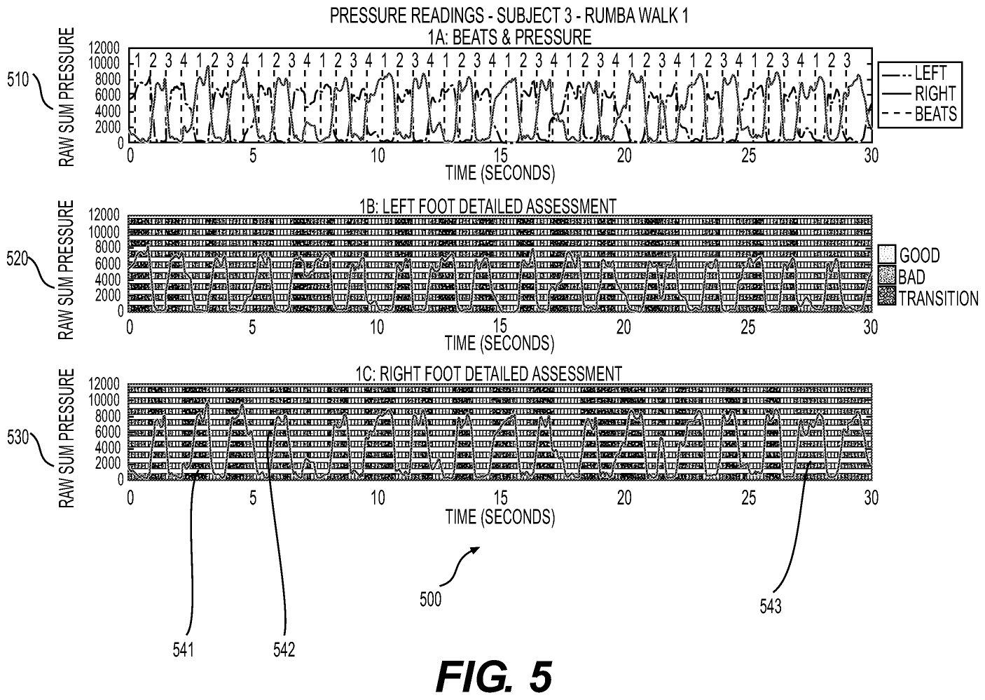

An example of the Dynamic Foot Pressure Analysis feedback (1040) is graphically represented in FIG. 5 (520,530). The green (542) color in the graphs identifies correct (610) dynamic foot pressure; the red (543) color identifies incorrect (630) dynamic foot pressure; the grey (541) color identifies when the user is lifting one foot off the ground and transferring the weight (620) to the other foot (520,530). The graphical representation of the Dynamic Foot Pressure Analysis feedback (1040) in FIG. 5 is represented textually in FIG. 6 as percentages (i.e., the number of data frames of the feedback divided by the total number of frames, multiplied by 100%) for each foot (641,642) for proper technique (610), transitions (620), and improper technique (630). The average (643) percentage of both feet for each dynamic foot pressure analysis feedback category is also computed for the overall dynamic foot pressure analysis score. The feedback is not limited to just one representation.

B. Rotational Movement Analysis

A Rotational Movement Analysis program (included in 1100) is designed to evaluate rotational movement of the vertebral column, at a pivot joint, or at a ball-and-socket joint, utilizing the processed video data (932) and the processed motion data (931) from cameras (480), three-dimensional inertial measurement sensing equipment, or reflective markers on the individual acquiring motion feedback (1150). Rotation is the only motion which occurs at pivot joints. Thus, pivot joints are uniaxial joints, joints where motion only occurs in a single plane. Examples of such joints are the proximal radioulnar joint, which allows the neck to rotate, and the atlantoaxial joint, which allows the the radius to rotate during pronation and supination movements of the forearm. Unlike pivot joints, ball-and-socket joints are multi-axial joints. Thus, at ball-and-socket joints, like the shoulder and hip, rotation is not the only motion which occurs at these joints.

By placing three-dimensional inertial measurement sensing equipment and/or reflective markers on the corresponding areas of the body where rotational movement occur, and cameras (480) in each axis, the processed motion data (1110) collected from these areas together with the captured video(s) (1120) at multiple angles will provide for the necessary input(s) for the Rotational Movement Analysis program to assess and compare (1140) the motion feedback (1150), wherein the motion feedback (1150) comprises: (i) the range and ability of rotational movement, and (ii) the quality of rotational movement depending on the movement at each given timestamp.

The step of comparing (1140) operates in a manner such that each individual motion datapoint (1111) of the timestamped motion data array(s) (1110) with computed angles and measurements (1130) from each individual video frame (1121) of the timestamped video data array(s) (1120) is compared against a set of video and movement model data stored in the central computing entity (150) that represents predetermined three-dimensional movement rules and constraints (1141), where said movement rules and constraints (1141) represent the ideal physical, three-dimensional movement for rotational movement for a given activity at a given moment in time during the movement.

C. Flexion and Extension Movement Analysis

The Flexion and Extension Movement Analysis program (included in 1100) is designed to evaluate movement which occurs within the sagittal plane and involves anterior or posterior movements of the body or limbs, utilizing the processed video data (932) and the processed motion data (931) from cameras (480), three-dimensional inertial measurement sensing equipment, or reflective markers on the individual acquiring motion feedback (1150). Areas of the body where flexion and extension occurs are the shoulder, hip, elbow, knee, wrist, metacarpophalangeal, metatarsophalangeal, and interphalangeal joints. Anterior bending of the head or vertebral column is flexion, while any posterior-going movement is extension.

In the limbs, flexion occurs when the joint bends or when the angle between bones decreases, while extension occurs when the joint straightens or when the angle increases between bones.

In the exemplary embodiment, by placing three-dimensional inertial measurement sensing equipment and/or reflective markers on the corresponding areas of the body where flexion and extension movement occur, and cameras (480) in each axis, the processed motion data (1110) collected from these areas together with the captured video(s) (1120) at multiple angles will provide for the necessary input(s) for the Flexion and Extension Movement Analysis program to assess and compare (1140) the motion feedback (1150), wherein the motion feedback (1150) comprises: (i) the range and ability of flexion and extension movement, and (ii) the quality of flexion and extension movement depending on the movement at each given timestamp.

The step of comparing (1140) operates in a manner such that each individual motion datapoint (1111) of the timestamped motion data array(s) (1110) with computed angles and measurements (1130) from each individual video frame (1121) of the timestamped video data array(s) (1120) is compared against a set of video and movement model data stored in the central computing entity (150) that represents predetermined three-dimensional movement rules and constraints (1141), where said movement rules and constraints (1141) represent the ideal physical, three-dimensional movement for flexion and extension movement for a given activity at a given moment in time during the movement.

D. Abduction, Adduction, and Circumduction Movement Analysis

The Abduction, Adduction, and Circumduction Analysis program (included in 1100) is designed to evaluate movement of the limbs, hands, fingers, or toes in the medial-lateral plane, utilizing the processed video data (932) and the processed motion data (931) from cameras (480), three-dimensional inertial measurement sensing equipment, or reflective markers on the individual acquiring motion feedback (1150). Areas of the body where abduction, adduction, and circumduction occurs are the shoulder, hip, wrist, metacarpophalangeal and metatarsophalangeal joints.

Abduction occurs when the limb moves laterally away from the midline of the body, while adduction occurs when the limb moves towards the body or across the midline. Abduction and adduction movements occur at condyloid, saddle, and ball-and-socket joints.

Circumduction is a rather interesting movement, because it involves the sequential combination of flexion, adduction, extension, and abduction at a joint. Circumduction is the movement of a body region in a circular fashion. Circumduction occur at biaxial condyloid, saddle, and at multi-axial ball-and-socket joints.

By placing three-dimensional inertial measurement sensing equipment and/or reflective markers on the corresponding areas of the body where abduction, adduction, and circumduction movement occur, and cameras (480) in each axis, the processed motion data (1110) collected from these areas together with the captured video(s) (1120) at multiple angles will provide for the necessary input(s) for the Abduction, Adduction, and Circumduction Movement Analysis program to assess and compare (1140) the motion feedback (1150), wherein the motion feedback (1150) comprises: (i) the range and ability of abduction, adduction, and circumduction movement, and (ii) the quality of abduction, adduction, and circumduction movement depending on the movement at each given timestamp.

The step of comparing (1140) operates in a manner such that each individual motion datapoint (1111) of the timestamped motion data array(s) (1110) with computed angles and measurements (1130) from each individual video frame (1121) of the timestamped video data array(s) (1120) is compared against a set video and movement model data stored in the central computing entity (150) that represents predetermined three-dimensional movement rules and constraints (1141), where said movement rules and constraints (1141) represent the ideal physical, three-dimensional movement for abduction, adduction, and circumduction movement for a given activity at a given moment in time during the movement.

E. Dorsiflexion and Plantar Flexion Movement Analysis

The Dorsiflexion and Plantar Flexion Movement Analysis program (included in 1100) is designed to evaluate movement at the ankle joint, a hinge joint, utilizing the processed video data (932) and the processed motion data (931) from cameras (480), three-dimensional inertial measurement sensing equipment, or reflective markers on the individual acquiring motion feedback (1150). The ankle joint only has two possible movements: dorsiflexion and plantar flexion. Dorsiflexion of the foot at the ankle joint moves the top of the foot toward the leg, while the plantar flexion lifts the heel and points the toes.

By placing the three-dimensional inertial measurement sensing equipment and/or reflective markers on the corresponding areas of the ankle joints, and cameras (480) in each axis, the processed motion data (1110) collected from these areas together with the captured video(s) (1120) at multiple angles will provide for the necessary input(s) for the Dorsiflexion and Plantar Flexion Movement Analysis program to assess and compare (1140) the motion feedback (1150), wherein the motion feedback (1150) comprises: (i) the range and ability of movement in the ankle joints, and (ii) the quality of movement in the ankle joints depending on the movement at each given timestamp.

The step of comparing (1140) operates in a manner such that each individual motion datapoint (1111) of the timestamped motion data array(s) (1110) with computed angles and measurements (1130) from each individual video frame (1121) of the timestamped video data array(s) (1120) is compared against a set of video and movement model data stored in the central computing entity (150) that represents predetermined three-dimensional movement rules and constraints (1141), where said movement rules and constraints (1141) represent the ideal physical, three-dimensional movement for dorsiflexion and plantar flexion movement for a given activity at a given moment in time during the movement.

F. Supination and Pronation Movement Analysis

An exemplary Supination and Pronation Movement Analysis program (included in 1100) is designed to evaluate movement of the forearm, utilizing the processed video data (932) and the processed motion data (931) from cameras (480), three-dimensional inertial measurement sensing equipment, or reflective markers on the individual acquiring motion feedback (1150). The forearm has two possible movements: supination and pronation. Pronation is movement that moves the forearm from the supinated (anatomical) position to the pronated (palm backward) position. Supination is the reverse movement.

By placing the three-dimensional inertial measurement sensing equipment and/or reflective markers on the corresponding areas of the forearms, and cameras (480) in each axis, the processed motion data (1110) collected from these areas together with the captured video(s) (1120) at multiple angles will provide for the necessary input(s) for the Supination and Pronation Movement Analysis program to assess and compare (1140) the motion feedback (1150), wherein the motion feedback (1150) comprises: (i) the range and ability of supination and pronation movement, and (ii) the quality of forearm movement depending on the movement at each given timestamp.

The step of comparing (1140) operates in a manner such that each individual motion datapoint (1111) of the timestamped motion data array(s) (1110) with computed angles and measurements (1130) from each individual video frame (1121) of the timestamped video data array(s) (1120) is compared against a set of video and movement model data stored in the central computing entity (150) that represents predetermined three-dimensional movement rules and constraints (1141), where said movement rules and constraints (1141) represent the ideal physical, three-dimensional movement for supination and pronation movement for a given activity at a given moment in time during the movement.

G. Protraction, Retraction, Depression, Elevation, Superior Rotation, and Inferior Rotation Movement Analysis

The exemplary Protraction, Retraction, Depression, Elevation, Superior Rotation, Inferior Rotation Movement Analysis program (included in 1100) is designed to evaluate the movement of the scapula, also known as the shoulder blade, utilizing the processed video data (932) and the processed motion data (931) from cameras (480), three-dimensional inertial measurement sensing equipment, or reflective markers on the individual acquiring motion feedback (1150). The scapula has six possible movements: Protraction, Retraction, Depression, Elevation, Superior Rotation, and Inferior Rotation.

Protraction and Retraction are anterior-posterior movements. Protraction occurs when the shoulder moves forward, while Retraction occurs when the shoulder is pulled posteriorly and medially toward the vertebral column.

Depression and Elevation are downward and upward movement of the scapula or, in layman terms, the shrugging of the shoulders.

Superior Rotation is a combination of Elevation and lateral rotation of the scapula away from the vertebral column. Superior Rotation is extremely vital for upper limb abduction. Without Superior Rotation, any abduction of the arm above shoulder height would not occur.

Inferior Rotation is a combination of Depression and medial rotation of the scapula toward the vertebral column. Inferior Rotation occurs during limb adduction.

By placing the three-dimensional inertial measurement sensing equipment and/or reflective markers on the corresponding areas of the scapulae, and cameras (480) in each axis, the processed motion data (1110) collected from these areas together with the captured video(s) (1120) at multiple angles will provide for the necessary input(s) for the Protraction, Retraction, Depression, Elevation, Superior Rotation, Inferior Rotation Movement Analysis program to assess and compare (1140) the motion feedback (1150), wherein the motion feedback (1150) comprises: (i) the range and ability of movement in the scapulae, and (ii) the quality of movement in the scapulae at each given timestamp.

The step of comparing (1140) operates in a manner such that each individual motion datapoint (1111) of the timestamped motion data array(s) (1110) with computed angles and measurements (1130) from each individual video frame (1121) of the timestamped video data array(s) (1120) is compared against a set of video and movement model data stored in the central computing entity (150) that represents predetermined three-dimensional movement rules and constraints (1141), where said movement rules and constraints (1141) represent the ideal physical, three-dimensional movement for protraction, retraction, depression, elevation, superior rotation, inferior rotation movement for a given activity at a given moment in time during the movement.

H. Inversion and Eversion Movement Analysis

The exemplary Inversion and Eversion Movement Analysis program (included in 1100) is novel and is designed to evaluate the movement of the multiple plane joints among the tarsal bones of the posterior foot (intertarsal joints), utilizing the processed video data (932) and the processed motion data (931) from cameras (480), three-dimensional inertial measurement sensing equipments, or reflective markers on the individual acquiring motion feedback (1150). There are two possible movements: inversion and eversion. Inversion occurs when the foot is turned inward toward the midline, and eversion occurs when the foot is turned out away from the midline. These movements are especially important, because these movements help to stabilize the foot when walking or running on uneven surfaces and in cutting movements during sports, such as soccer.

By placing the three-dimensional inertial measurement sensing equipment and/or reflective markers on the corresponding areas of the foot where inversion and eversion movement occur, and cameras (480) in each axis, the processed motion data (1110) collected from these areas together with the captured video(s) (1120) at multiple angles will provide for the necessary input(s) for the Inversion and Eversion Movement Analysis program to assess and compare (1140) the motion feedback (1150), wherein the motion feedback (1150) comprises: (i) the range and ability of inversion and eversion, and (ii) the quality of inversion and eversion depending on the movement at each given timestamp.

The step of comparing (1140) operates in a manner such that each individual motion datapoint (1111) of the timestamped motion data array(s) (1110) with computed angles and measurements (1130) from each individual video frame (1121) of the timestamped video data array(s) (1120) is compared against a set of video and movement model data stored in the central computing entity (150) that represents predetermined three-dimensional movement rules and constraints (1141), where said movement rules and constraints (1141) represent the ideal physical, three-dimensional movement for inversion and eversion movement for a given activity at a given moment in time during the movement.

I. Timing Analysis

When movement is accompanied with music, the Timing Analysis program (1200) of FIG. 12 is designed to evaluate and compare (1240) combinations of correct dynamic foot pressure movement (1242), correct movements from categories (B-H) above (1243) (including correct rotational movement, correct flexion and extension movement, correct abduction, correct adduction, correct circumduction movement, correct dorsiflexion and plantar flexion, correct supination and pronation movement, correct inversion and eversion movement, and correct movement of the scapula), at each timestamp for the movement data (1242, 1243) in correlation with the beat (1230) of the audio sample of music (1210), utilizing all of the processed data from the timestamped pressure data array(s) (913), the timestamped video data array(s) (912), and the motion data array(s) (911).

The timing analysis differs completely from the other analysis categories, because this category is specifically for dance movement(s) and this category of analysis is dependent on the feedback (1242, 1243) of the other movement analysis categories (A-H). For dance movement, defining timing (1250) simply as matching foot strike to the beat (1230) of an audio sample (1210) of music is not sufficient enough for the individual to improve their ability to match their movement in accordance to the beat (1230) and rhythm of an audio sample (1210) of music.

If both movement (1242,1243) is correct at a specific timestamp, and the timestamp of the movement (1242,1243) matches that of the timestamp of the individual beat (1230) from the audio sample (1210) of music from the movement video (141), then the rule (1241) for correct timing (1250) at that specific timestamp and beat (1230) is true.

The beats (1230) and other music information of the extracted (922) audio sample (1210) from the processed video data array(s) (912) is extracted using beat detection algorithms and other music information retrieval tools (1220) stored in the central computing entity (150).

An example of a graphical representation (500) of the timing analysis comparison (1240) is shown in FIG. 5. The extracted (1220) beats (1230) are laid on top of the left and right pressure data (510). The timing feedback (1250) in FIG. 5 (510) is assessed from the comparison (1240) of the beat (1230) to the dynamic pressure feedback results (1242) at each given timestamp.

An individual's timing results (1250) can be expressed as a numerical ratio of correct, synchronized dance movements to the beats (1230) of the audio sample (1210) of music from the video (141) over the total number of beats (1230) in the audio sample (1210) of the music, in addition to the formats and representations described in Step 6 below.

Step 5. Results Transferred to Smart Device

After the above analyses have been performed by the central computing entity (150), the results (1320) of the analysis (940) are transferred (1310) to the user's smart device for feedback display (710) or to any other user with access of the user's movement results (1320) through the communications channel. A suitable communications system, such as the Internet, has been discussed previously.

Step 6. Displaying Results

FIG. 13 is a flowchart that shows an exemplary process (1300) for displaying results of the movement analysis system of the present invention. And FIG. 7 shows a sample display (700) of the movement feedback results delivered by the movement analysis system of the present invention. After the results (1320) of the movement feedback analysis (940) have been transferred (1310) from the central computing entity (150) to the user's displaying smart device (710), the displaying smart device (710) displays (1330) the results (1320) in an easy to understand format, as shown in FIG. 7. The results (1320) will include information such as the data log title, timestamp, and synchronized, detailed, and color-coded annotations (731,723), animations (720,730), and comments (734) of movement and audio feedback (723) on the scrubber (722) layered over the video (720). The individual or any other individual with access to the individual's analyzed results (1320) will then have the option to select and go back (740) to additional view(s) (1340,1350,1360) and subset displays (732,1341) of the video playback (721) and feedback results (1320), annotations (1320), and comments (1320), to gain a better understanding of the individual's movements in order to better the movement, and to show the individual's progress over time (1351) and the progress over time compared to other individuals (1361). The results (1320) of the movement feedback analysis (940) will be displayed (1330) in both a textual and graphical format upon the displaying smart device (710). Any individual with access to a user's movement feedback results (1320) will have the ability to post comments (733) at a specific timestamp of the movement feedback results (1320). Also, the feedback (1320) may be presented as a particular color (731,723) signifying a specific result.

The above described analysis tool significantly improves the analysis of physical motion and the overall learning process for learning proper movement. Indeed, replaying the synchronized signals provides a valuable teaching tool in that a user can visualize their motion and the feedback provided to them. Providing the combination of these signals removes guesswork associated with trying to pinpoint the problem areas and the degree to which they are a problem. Additionally, the present invention relates to many improvements in the learning process, such as combining numerous signals (video, audio, motion capture, pressure, etc.), allowing for numerous display options, and numerous playback options.

As to a further discussion of the manner of usage of the present invention, the same should be apparent from the above description. Accordingly, no further discussion relating to the manner of usage will be provided.

With respect to the above description then, it is to be realized that the optimum relationships for the components of the invention, to include variations in proportions and manner of use are deemed readily apparent and obvious to one skilled in the art.

Therefore, the foregoing is considered as illustrative only of the principles of the invention. Further, since numerous modifications and changes will readily occur to those skilled in the art, it is not desired to limit the invention to the exact composition and use shown and described, and accordingly, all suitable modifications and equivalents may be resorted to, falling within the scope of the invention.

I claim:

1. A movement analysis system for providing a quantitative movement assessment, comprising:
a data capture system to record and capture video, audio, pressure data, and motion data during an individual's movement;
a synchronization module wherein captured video, motion data, and pressure data is synchronized with audio using timestamps from a common timebase in the audio, and wherein the synchronized video, audio, motion data, and pressure data is transmitted through a communication system;
a central computing entity comprising:
a memory, wherein instructions are stored; and
a processor for executing the stored instructions and configured to:
receive the transmitted data from the communication system in a database;
conduct a movement analysis of the received data, wherein the received data is compared to model data;
determine results of the movement analysis, wherein the results contain visual data; and
transfer the results to a smart device, wherein the results are displayed on a display screen of the smart device.

2. The movement analysis system of claim 1, wherein the processor is further configured to analyze dynamic foot pressure utilizing the received pressure data and the received video of the individual's movement.

3. The movement analysis system of claim 1, wherein the processor is further configured to analyze rotational movement utilizing three-dimensional inertial measurement data, reflective marker data, and the received video of the individual's movement.

4. The movement analysis system of claim 1, wherein the processor is further configured to analyze flexion and extension movement utilizing three-dimensional inertial measurement data, reflective marker data, and the received video of the individual's movement.

5. The movement analysis system of claim 1, wherein the processor is further configured to analyze abduction, adduction, and circumduction movement using three-dimensional inertial measurement data, reflective marker data, and the received video of the individual's movement.

6. The movement analysis system of claim 1, wherein the processor is further configured to analyze dorsiflexion and plantar flexion movement utilizing three-dimensional inertial measurement data, reflective marker data, and the received video of the individual's movement.

7. The movement analysis system of claim 1, wherein the processor is further configured to analyze supination and pronation movement utilizing three-dimensional inertial measurement data, reflective marker data, and the received video of the individual's movement.

8. The movement analysis system of claim 1, wherein the processor is further configured to analyze protraction, retraction, depression, elevation, superior rotation, and inferior rotation movement utilizing three-dimensional inertial measurement data, reflective marker data, and the received video of the individual's movement.

9. The movement analysis system of claim 1, wherein the processor is further configured to analyze inversion and eversion movement utilizing three-dimensional inertial measurement data, reflective marker data, and the received video of the individual's movement.

10. The movement analysis system of claim 1, wherein the processor is further configured to analyze timing utilizing pressure data, three-dimensional inertial measurement data, and reflective marker data synchronized with the audio captured with the received video.

11. The movement analysis system of claim 1, wherein the processor is further configured to perform a movement assessment including generating data for a visual display of synchronized, detailed, and color-coded graphical and textual annotations, animations, and comments of the movement feedback results layered over the received video of the individual's movement.

12. The movement analysis system of claim 11, wherein the movement assessment includes comments regarding information of an individual's data log.

13. The movement analysis system of claim 11, wherein a movement assessment includes generating data for a visual display of the individual's progress over time.

14. The movement analysis system of claim 11, wherein the movement assessment includes a visual display of numerical scores for each category of feedback analysis for an individual.

15. The movement analysis system of claim 11, wherein a movement assessment includes a visual display of an individual's progress over time against other individuals.

16. The movement analysis system of claim 11, wherein a movement assessment includes a visual display comparing numerical scores for each category of feedback analysis of at least two individuals.

17. The movement analysis system of claim 1, wherein the processor is further configured to communicate an individual's movement feedback to a second smart device where the results are displayed.

18. The movement analysis system of claim 12, wherein the movement assessment includes comments which are created using an input device by a user who has access to an individual's movement feedback results.

19. A movement analysis method for providing a quantitative movement assessment, comprising the steps of:
(a) recording and capturing video, pressure data, and motion data during an individual's movement training;
(b) synchronizing captured video, pressure data and motion data with audio using timestamps from a common timebase in the audio;
(c) transferring the collected data to a central computing entity through a communication system;
(d) receiving the transferred data into a central computing entity database;
(e) conducting a movement analysis of the individual using the transferred data wherein the movement analysis comprises:
a comparison of the received synchronized foot pressure data points with model foot pressure data points;
an analysis of rotational movement; and
a timing analysis;
(f) determining results of the movement analysis;
(g) communicating the results to a smart device; and
(h) displaying the results upon a display monitor of the smart device.

20. The movement analysis method of claim 19, wherein the results of the movement analysis includes generating data for a visual display of synchronized, detailed, and color-coded graphical and textual annotations, animations, and comments of movement feedback results layered over the received video of the individual's movement.
